SEQUENCE LISTING (16 sequences)

SEQ ID NO: 1 - DNA, 3891 bp, Clostridium botulinum
atgccatttgttaataaacaatttaattataaagatcctgtaaatggtgttgatattgcttatataaaaattccaaatgcaggacaaatgcaaccagtaaaagcttttaaaattcataataaaatatgggttattccagaaagagatacatttacaaatcctgaagaaggagatttaaatccaccaccagaagcaaaacaagttccagtttcatattatgattcaacatatttaagtacagataatgaaaaagataattatttaaagggagttacaaaattatttgagagaatttattcaactgatcttggaagaatgttgttaacatcaatagtaaggggaataccattttggggtggaagtacaatagatacagaattaaaagttattgatactaattgtattaatgtgatacaaccagatggtagttatagatcagaagaacttaatctagtaataataggaccctcagctgatattatacagtttgaatgtaaaagctttggacatgaagttttgaatcttacgcgaaatggttatggctctactcaatacattagatttagcccagattttacatttggttttgaggagtcacttgaagttgatacaaatcctcttttaggtgcaggcaaatttgctacagatccagcagtaacattagcacatgaacttatacatgctggacatagattatatggaatagcaattaatccaaatagggtttttaaagtaaatactaatgcctattatgaaatgagtgggttagaagtaagctttgaggaacttagaacatttgggggacatgatgcaaagtttatagatagtttacaggaaaacgaatttcgtctatattattataataagtttaaagatatagcaagtacacttaataaagctaaatcaatagtaggtactactgcttcattacagtatatgaaaaatgtttttaaagagaaatatctcctatctgaagatacatctggaaaattttcggtagataaattaaaatttgataagttatacaaaatgttaacagagatttacacagaggataattttgttaagttttttaaagtacttaacagaaaaacatatttgaattttgataaagccgtatttaagataaatatagtacctaaggtaaattacacaatatatgatggatttaatttaagaaatacaaatttagcagcaaactttaatggtcaaaatacagaaattaataatatgaattttactaaactaaaaaattttactggattgtttgaattttataagttgctatgtgtaagagggataataacttctaaaactaaatcattagataaaggatacaataaggcattaaatgatttatgtatcaaagttaataattgggacttgttttttagtccttcagaagataattttactaatgatctaaataaaggagaagaaattacatctgatactaatatagaagcagcagaagaaaatattagtttagatttaatacaacaatattatttaacctttaattttgataatgaacctgaaaatatttcaatagaaaatctttcaagtgacattataggccaattagaacttatgcctaatatagaaagatttcctaatggaaaaaagtatgagttagataaatatactatgttccattatcttcgtgctcaagaatttgaacatggtaaatctaggattgctttaacaaattctgttaacgaagcattattaaatcctagtcgtgtttatacatttttttcttcagactatgtaaagaaagttaataaagctacggaggcagctatgtttttaggctgggtagaacaattagtatatgattttaccgatgaaactagcgaagtaagtactacggataaaattgcggatataactataattattccatatataggacctgctttaaatataggtaatatgttatataaagatgattttgtaggtgctttaatattttcaggagctgttattctgttagaatttataccagagattgcaatacctgtattaggtacttttgcacttgtatcatatattgcgaataaggttctaaccgttcaaacaatagataatgctttaagtaaaagaaatgaaaaatgggatgaggtctataaatatatagtaacaaattggttagcaaaggttaatacacagattgatctaataagaaaaaaaatgaaagaagctttagaaaatcaagcagaagcaacaaaggctataataaactatcagtataatcaatatactgaggaagagaaaaataatattaattttaatattgatgatttaagttcgaaacttaatgagtctataaataaagctatgattaatataaataaatttttgaatcaatgctctgtttcatatttaatgaattctatgatcccttatggtgttaaacggttagaagattttgatgctagtcttaaagatgcattattaaagtatatatatgataatagaggaactttaattggtcaagtagatagattaaaagataaagttaataatacacttagtacagatataccttttcagctttccaaatacgtagataatcaaagattattatctacatttactgaatatattaagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3876 bp, Clostridium botulinum
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaagctccaggaatatgtattgatgttgataatgaagatttgttctttatagctgataaaaatagtttttcagatgatttatctaaaaacgaaagaatagaatataatacacagagtaattatatagaaaatgacttccctataaatgaattaattttagatactgatttaataagtaaaatagaattaccaagtgaaaatacagaatcacttactgattttaatgtagatgttccagtatatgaaaaacaacccgctataaaaaaaatttttacagatgaaaataccatctttcaatatttatactctcagacatttcctctagatataagagatataagtttaacatcttcatttgatgatgcattattattttctaacaaagtttattcatttttttctatggattatattaaaactgctaataaagtggtagaagcaggattatttgcaggttgggtgaaacagatagtaaatgattttgtaatcgaagctaataaaagcaatactatggataaaattgcagatatatctctaattgttccttatataggattagctttaaatgtaggaaatgaaacagctaaaggaaattttgaaaatgcttttgagattgcaggagccagtattctactagaatttataccagaacttttaatacctgtagttggagcctttttattagaatcatatattgacaataaaaataaaattattaaaacaatagataatgctttaactaaaagaaatgaaaaatggagtgatatgtacggattaatagtagcgcaatggctctcaacagttaatactcaattttatacaataaaagagggaatgtataaggctttaaattatcaagcacaagcattggaagaaataataaaatacagatataatatatattctgaaaaagaaaagtcaaatattaacatcgattttaatgatataaattctaaacttaatgagggtattaaccaagctatagataatataaataattttataaatggatgttctgtatcatatttaatgaaaaaaatgattccattagctgtagaaaaattactagactttgataatactctcaaaaaaaatttgttaaattatatagatgaaaataaattatatttgattggaagtgcagaatatgaaaaatcaaaagtaaataaatacttgaaaaccattatgccgtttgatctttcaatatataccaatgatacaatactaatagaaatgtttaataaatataatagcgaaattttaaataatattatcttaaatttaagatataaggataataatttaatagatttatcaggatatggggcaaaggtagaggtatatgatggagtcgagcttaatgataaaaatcaatttaaattaactagttcagcaaatagtaagattagagtgactcaaaatcagaatatcatatttaatagtgtgttccttgattttagcgttagcttttggataagaatacctaaatataagaatgatggtatacaaaattatattcataatgaatatacaataattaattgtatgaaaaataattcgggctggaaaatatctattaggggtaataggataatatggactttaattgatataaatggaaaaaccaaatcggtattttttgaatataacataagagaagatatatcagagtatataaatagatggttttttgtaactattactaataatttgaataacgctaaaatttatattaatggtaagctagaatcaaatacagatattaaagatataagagaagttattgctaatggtgaaataatatttaaattagatggtgatatagatagaacacaatttatttggatgaaatatttcagtatttttaatacggaattaagtcaatcaaatattgaagaaagatataaaattcaatcatatagcgaatatttaaaagatttttggggaaatcctttaatgtacaataaagaatattatatgtttaatgcggggaataaaaattcatatattaaactaaagaaagattcacctgtaggtgaaattttaacacgtagcaaatataatcaaaattctaaatatataaattatagagatttatatattggagaaaaatttattataagaagaaagtcaaattctcaatctataaatgatgatatagttagaaaagaagattatatatatctagatttttttaatttaaatcaagagtggagagtatatacctataaatattttaagaaagaggaagaaaaattgtttttagctcctataagtgattctgatgagttttacaatactatacaaataaaagaatatgatgaacagccaacatatagttgtcagttgctttttaaaaaagatgaagaaagtactgatgagataggattgattggtattcatcgtttctacgaatctggaattgtatttgaagagtataaagattatttttgtataagtaaatggtacttaaaagaggtaaaaaggaaaccatataatttaaaattgggatgtaattggcagtttattcctaaagatgaagggtggactgaataa

SEQ ID NO: 4 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 5 - DNA, 3843 bp, Clostridium botulinum
atgccaataacaattaacaactttaattattcagatcctgttgataataaaaatattttatatttagatactcatttaaatacattagctaatgagcctgaaaaagcctttcgcattatagggaatatatgggtaatacccgatagattttcaagagattctaatccaaatttaaataaacctcctcgagttacaagccctaaaagtggttattatgatcctaattatttgagtactgattctgaaaaagatacatttttaaaagaaattataaagttatttaaaagaattaactctagagaaataggagaagaattaatatatagacttgcaacagacataccctttcctgggaataacaatactccaattaatacttttgattttgatgtagattttaacagtgttgatgttaaaactagacaaggtaacaactgggttaaaactggtagtataaatcctagtgttataataactggacctagagaaaacattatagacccagaaacttctacgtttaaattaactaacaatacttttgcggcacaagaaggatttggtgctttatcaataatttcaatatcacctagatttatgctaacatatagtaatgcaactaataatgtaggagagggtagattttctaagtctgaattttgcatggatccaatactaattttaatgcatgaacttaatcatgcaatgcataatttatatggaatagctataccaaatgatcaaagaatttcatctgtaactagtaatattttttattctcaatataaggtgaaattagagtatgcagaaatatatgcatttggaggtccaactatagaccttattcctaaaagtgcaaggaaatattttgaggaaaaggcattggattattatagatccatagctaaaagacttaatagtataactactgcaaatccttcaagctttaataaatatataggagaatataaacagaaacttattagaaagtatagattcgtagtagaatcttcaggtgaagttgcagtagatcgtaataagtttgctgagttatataaagaacttacacaaatatttacagaatttaactacgctaaaatatataatgtacaaaataggaaaatatatctttcaaatgtatatactccggttacggcaaatatattagacgataatgtttatgatatacaaaatggatttaacatacctaaaagtaatttaaatgtactatttatgggtcaaaatttatctcgaaatccagcattaagaaaagtcaatcctgaaaatatgctttatttatttacaaaattttgccataaagcaatagatggtagatcattatataataaaacattagattgtagagagcttttagttaaaaatactgacttaccctttataggtgatattagtgatatcaaaactgatatatttttaagcaaagatattaatgaagaaactgaagttatagactatccggacaatgtttcagtggatcaagttattctcagtaagaatacctcagaacatggacaactagatttattataccctattattgaaggtgagagtcaagtattaccgggagagaatcaagtcttttatgataatagaactcaaaatgttgattatttgaattcttattattacctagaatctcaaaaactaagtgataatgttgaagattttacttttacgacatcaattgaggaagctttggataatagtggaaaagtatatacttactttcctaaactagctgataaagtaaatacgggtgttcaaggtggtttatttttaatgtgggcaaatgatgtagttgaagattttactacaaatattctaagaaaagatacattagataaaatatcagatgtatcagctattattccctatataggacctgcattaaatataagtaattctgtaagaaggggaaattttactgaagcatttgcagttaccggtgtaactattttattagaagcgtttcaagaatttacaatacctgcacttggtgcatttgtgatttatagtaaggttcaagaaagaaacgagattattaaaactatagataattgtttagaacaaaggattaaaagatggaaagattcatatgaatggatgataggaacgtggttatccaggattactactcaatttaataatataagttatcaaatgtatgattctttaaattatcaggcagatgcaatcaaagataaaatagatttagaatataaaaaatactcaggaagtgataaagaaaatataaaaagtcaagttgaaaatttaaaaaatagtttagatataaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatataattaatgaatatttcaatagtattaatgattcaaaaattttgagcttacaaaacaaaaaaaatgctttagtggatacatcaggatataatgcagaagtgaggctagaaggtgatgttcaagttaatacgatatatacaaatgattttaaattaagtagttcaggagataaaattatagtaaatttaaataataatattttatatagcgctatttatgagaactctagtgttagtttttggattaagatatctaaagatttaactaattctcataatgaatatacaataattaatagtataaaacaaaattctgggtggaaattatgtattaggaatggcaatatagaatggattttacaagatattaatagaaagtataaaagtttaatttttgattatagtgaatcattaagtcatacaggatatacaaataaatggttttttgttactataactaataatataatggggtatatgaaactttatataaatggagaattaaagcagagtgaaagaattgaagatttaaatgaggttaagttagataaaaccatagtatttggaatagatgagaatatagatgagaatcagatgctttggattagagattttaatattttttctaaagaattaagcaatgaagatattaatattgtatatgagggacaaatattaagaaatgttattaaagattattggggaaatcctttgaagtttgatacagaatattatattattaatgataattatatagataggtatatagcacctaaaagtaatatacttgtacttgttcagtatccagatagatctaaattatatactggaaatcctattactattaaatcagtatctgataagaatccttatagtagaattttaaatggagataatataatgtttcatatgttatataatagtgggaaatatatgataataagagatactgatacaatatatgcaatagaaggaagagagtgttcaaaaaattgtgtatatgcattaaaattacagagtaatttaggtaattatggtataggtatatttagtataaaaaatattgtatctcaaaataaatattgtagtcaaattttctctagttttatgaaaaatacaatgcttctagcagatatatataaaccttggagattttcttttgaaaatgcatacacgccagttgcagtaactaattatgagacaaaactattatcaacttcatctttttggaaatttatttctagggatccaggatgggtagagtaa

SEQ ID NO: 6 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYKVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 7 - DNA, 3858 bp, Clostridium botulinum
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcctagacctacttcaaagtatcaaagttattatgatcctagttatttatctactgatgagcaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattgtatggaataaatataccatctgataaaaggattcgtccacaagttagcgagggatttttttctcaagatggacccaacgtacaatttgaggaattatacacatttggaggatcagatgttgaaataatacctcaaattgaaagattacaattaagagaaaaagcattaggtcactataaagatatagcgaaaagacttaataatattaataaaactattccttctagttggagtagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaagcattatctacctgtatttgcaaatatattagatgataatatttatactataataaacggttttaatttaacaactaaaggttttaatatagaaaattcgggtcagaatatagaaaggaatcctgcactacaaaaacttagttcagaaagtgtagtagatttgtttacaaaagtatgtttaagattaacaagaaatagtagagatgattcaacatgtattcaagttaaaaataatacattaccttatgtagctgataaagatagcatttcacaagaaatatttgaaagtcaaattattacagatgagactaatgtagaaaattattcagataatttttcattagatgaatctattttagatgcaaaagtccctactaatcctgaagcagtagatccactgttacccaatgttaatatggaacctttaaatgttccaggtgaagaagaagtattttatgatgatattactaaagatgttgattatttaaactcttattattatttggaagcccaaaaattaagtaataatgttgaaaatattactcttacaacttcagttgaagaagcattaggttatagcaataagatatacacatttttacctagcttagctgaaaaagtgaataaaggtgttcaagcaggtttattcttaaattgggcgaatgaagtagttgaggattttactacaaatattatgaaaaaagatacattggataaaatatcagatgtatcagccataattccatatataggacctgccttaaatataggaaattcagcattaaggggaaactttaagcaagcatttgcaacagctggtgtagcttttttgttagaaggatttccagagtttacaatacctgcactcggtgtatttaccttttatagttctattcaagaaagagagaaaattattaaaactatagaaaattgtttagaacaaagagttaagagatggaaagattcatatcaatggatggtatcaaattggttgtcaagaattactactcgatttaatcatataagttatcaaatgtatgattctttgagttatcaggcagatgcaatcaaagctaaaatagatttagaatataaaaaatactcaggaagtgataaagaaaatataaaaagtcaagttgaaaatttaaaaaatagtttagatgtaaaaatctcggaagcaatgaataatataaataaatttatacgagaatgttctgtaacatacttatttaaaaatatgctccctaaagtaattgatgaattaaataagtttgatttaaaaactaaaacagaattaattaatcttatagatagtcataatattattctagttggtgaagtagatagattaaaagcaaaagtaaatgagagttttgaaaatacaataccctttaatattttttcatatactaataattctttattaaaagatatgattaatgaatatttcaatagtattaatgattcaaaaattttgagcttacaaaataaaaaaaatactttgatggatacatcaggatataacgcagaagtgagagtagaaggcaatgttcagcttaatccaatatttccatttgactttaaattaggtagttcaggggatgatagaggtaaagttatagtaacccagaatgaaaatattgtatataatgctatgtatgaaagttttagtattagtttttggattaggataaataaatgggtaagtaatttacctggatatactataattgatagtgttaaaaataactcaggttggagtataggtattattagtaattttttagtgtttactttaaaacaaaatgaaaatagtgaacaagatataaactttagttatgatatatcaaagaatgctgcgggatataataaatggttttttgtaactattactaccaatatgatgggaaatatgatgatttatataaatggaaaattaatagatactataaaagttaaagagttaactggaattaattttagcaaaactataacatttcaaatgaataaaattccaaatactggcttaattacctcagattctgataacatcaatatgtggataagggatttttatatctttgctaaagaattagatgataaagatattaatatattatttaatagcttgcaatatactaatgttgtaaaagattattggggaaatgatttaagatatgataaagaatattacatgattaacgtaaattatatgaatagatatatgtctaaaaaaggcaatggaattgtttttaatacacgtaaaaataataatgacttcaatgaaggatataaaattataataaaaagaattagaggaaatacaaatgatactagagtacgaggagaaaatgtattatattttaatactacaattgataacaaacaatatagtttaggtatgtataaaccttctagaaatctagggactgatttagttccactaggtgcattggatcaaccaatggatgagatacgtaaatatggttcgtttataatacaaccatgcaatacttttgattactatgcatcacaattatttttgtcaagtaatgcaacaacaaataggcttggaatactatcaattggtagttatagtttcaaacttggagatgactattggtttaatcacgaatatttaattcctgttataaaaatagagcattatgcttcattattagaatcaacatcaactcattgggtttttgtacctgcaagtgaataa

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTRFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 9 - DNA, 3756 bp, Clostridium botulinum
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcaccgttttggatcaatagctatagtaacattctcacctgaatattcttttagatttaatgataattgtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttacgaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaaataataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattcgaagataatcgaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaattgtacaatgaattttaaaaataataatggaaataatattgggttgttaggtttcaaggcagatactgtcgttgctagtacttggtattatacacatatgagagatcatacaaacagcaatggatgtttttggaactttatttctgaagaacatggatggcaagaaaaataa

SEQ ID NO: 10 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 11 - DNA, 3843 bp, Clostridium botulinum
atgccagttgtaataaatagttttaattataatgaccctgttaatgatgagacaattttatacatgcagaaaccatatgaagaaagaagtagaaaatattataaagcttttgagattatgcctaatgtttggataatgcctgagagagatacaataggaactaagcctgatgagtttcaggtgccggattcattaaagaacggaagtagtgcttattatgatcctaattatttaaccactgatgctgaaaaagatagatatttaaaaacaatgataaaattatttaatagaattaatagtaatcctacagggaaagttttgttagaagaagtatcaaatgctagaccatatttaggagatgatgacacgctaattaatgaattccttccagttaatgtaactacaagtgttaatataaaattttcaactgatgttgaaagttcaataatatcgaatcttcttgtattgggagcaggacctgatatatttaaagcttactgtaccccccttgtaaggtttaataagtcagataaattaattgaaccaagtaatcatggttttggatcaattaatatcttgacattttcacctgagtatgaacatatttttaatgatattagtggagggaatcataatagtacagaatcatttattgcagatcctgcaatttcactagctcatgaattgatacatgcactacatggattatacggggctaaggcagttactcataaagagtctctagtagcagagcgaggacctcttatgatagccgaaaagcccataaggctagaagaatttttaacttttggaggtgaggatttaaatatcattcctagtgctatgaaggaaaaaatatataacgatcttttagctaactatgaaaaaatagctactagacttagagaagttaatacggctcctcctggatatgatattaatgaatataaagattattttcaatggaagtatggactagatagaaatgcagatggaagttatactgtgaatagaaataaatttaatgaaatttataaaaaattatatagctttacagagattgacttagcaaataaatttaaagtaaaatgtagaaatacttattttattaaatatggatttgtaaaagttccaaatttgttagatgatgatatttatactgtatcagaggggtttaatataggtaatttagcagtaaacaatcgcggacaaaatataaatttaaatcctaaaattattgattccattccagataaaggtttagtggaaaagattattaaattttgtaagagcattattcctagaaaaggtacgaagcagtcaccgtcactatgcattagagtaaataatagggagttattttttgtagcttcagaaagtagctataatgaaagtgatattaatacacctaaagaaattgacgatacaacaaatctaaataataattatagaaataatttagatgaagttattttagattataatagtgagacaatacctcaaatatcaaatcgaacattaaatacacttgtacaagacaatagttatgtgccaagatatgattctaatggaacaagtgaaatagaggaatatgatgttgttgactttaatgtatttttctatttacatgcacaaaaagtaccagaaggtgaaaccaatataagtttaacttcttcaattgatacagcattattagaagaatccaaagtatatacatttttttcttcagagtttatcgatactatcaataaacctgtaaatgcagcactatttatagattggataagcaaagtaataagagattttaccactgaagctacacaaaaaagtactgttgataagattgcagacatatctttaattgtaccctatgtaggtcttgctttgaatatagttattgaggcagaaaaaggaaattttgaggaggcatttgaattattaggagcgggtattttattagaatttgtgccagagcttacaattcctgtaattttagtgtttacgataaaatcctatatagattcatatgagaataaaaataaagcaattaaagcaataaataattcattaatcgaaagagaagcaaagtggaaagaaatatatagttggatagtatcaaattggcttactagaattaatacgcaatttaataaaagaaaagagcaaatgtatcaggctttacaaaatcaagtagatgcaataaaaacagcaatagaatataaatataataattatacttcagatgagaaaaatagacttgaatctaaatataatatcaataatatagaagaagaattgaataaaaaagtttctttagcaatgaaaaatatagaaagatttatgacagaaagttctatatcttatttaatgaaattaataaatgaagccgaagttggtaaattaaaagaatatgataaacatgttaagagcgatttattagactatattctctaccataaattaatcttaggagagcagacaaaggaattaattgatttggtgactagtactttgaatagtagtattccatttgaactttcttcatatactaatgataaaattctaattatatattttaatagattatataaaaaaattaaagatagttctattttagatatgcgatatgaaaataataaatttatagatatctctggatatggttcaaatataagcattaatggaaacgtatatatttattcaacaaatagaaatcaatttggaatatatagtggtaggcttagtgaagttaatatagctcaaaataatgatattatatacaatagtagatatcaaaattttagtattagtttctgggtaaccattcctaaacactacagacctatgaatcgtaatcgggaatacactataataaattgtatggggaataataattcgggatggaaaatatcacttagaactattagagattgtgaaataatttggactttacaagatacttccggaaataaggaaaaattaatttttaggtatgaagaacttgctagtatatctgattatataaataaatggatttttgtaactattactaataatagattaggcaattctagaatttacatcaatggaaatttaatagttgaaaaatcaatttcgaatttaggtgatattcatgttagtgataatatattatttaaaattgttggttgtgatgatgaaacgtatgttggtataagatattttaaagtttttaatacggaattagataaaacagaaattgagactttatatagtaatgagccagatccaagtatcttaaaagactattggggaaattatttgctatataataaaaaatattatttattcaatttactaagaaaagataagtatattactcggaattcaggcattttaaatattaatcaacaaagaggtgttactggaggcatatctgtttttttgaactataaattatatgaaggagtagaagttattataagaaaaaatgctcctatagatatatctaatacagataattttgttagaaaaaacgatctagcatacattaatgtagtagatcatggtgtagaatatcggttatatgctgatatatcaattacaaaatcagagaaaataataaaattaataagaacatctaatccaaacgatagcttaggtcaaattatagttatggattcaataggaaataattgcacaatgaattttcaaaacaatgatgggagcaatataggattactaggttttcattcagatgatttggttgctagtagttggtattataaccatatacgaagaaacactagcagtaatggatgcttttggagttttatttctaaagagcatggttggaaagaataa

SEQ ID NO: 12 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 13 - DNA, 3894 bp, Clostridium botulinum
  misc_feature (20)..(20): n is a, c, g, or t
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaaaataccggtaaatctgaacagtgtattattgttaataatgaggatttatttttcatagctaataaagatagtttttcaaaagatttagctaaagcagaaactatagcatataatacacaaaataatactatagaaaataatttttctatagatcagttgattttagataatgatttaagcagtggcatagacttaccaaatgaaaacacagaaccatttacaaattttgacgacatagatatccctgtgtatattaaacaatctgctttaaaaaaaatttttgtggatggagatagcctttttgaatatttacatgctcaaacatttccttctaatatagaaaatctacaactaacgaattcattaaatgatgctttaagaaataataataaagtctatacttttttttctacaaaccttgttgaaaaagctaatacagttgtaggtgcttcactttttgtaaactgggtaaaaggagtaatagatgattttacatctgaatccacacaaaaaagtactatagataaagtttcagatgtatccataattattccctatataggacctgctttgaatgtaggaaatgaaacagctaaagaaaattttaaaaatgcttttgaaataggtggagccgctatcttaatggagtttattccagaacttattgtacctatagttggattttttacattagaatcatatgtaggaaataaagggcatattattatgacgatatccaatgctttaaagaaaagggatcaaaaatggacagatatgtatggtttgatagtatcgcagtggctctcaacggttaatactcaattttatacaataaaagaaagaatgtacaatgctttaaataatcaatcacaagcaatagaaaaaataatagaagatcaatataatagatatagtgaagaagataaaatgaatattaacattgattttaatgatatagattttaaacttaatcaaagtataaatttagcaataaacaatatagatgattttataaaccaatgttctatatcatatctaatgaatagaatgattccattagctgtaaaaaagttaaaagactttgatgataatcttaagagagatttattggagtatatagatacaaatgaactatatttacttgatgaagtaaatattctaaaatcaaaagtaaatagacacctaaaagacagtataccatttgatctttcactatataccaaggacacaattttaatacaagtttttaataattatattagtaatattagtagtaatgctattttaagtttaagttatagaggtgggcgtttaatagattcatctggatatggtgcaactatgaatgtaggttcagatgttatctttaatgatataggaaatggtcaatttaaattaaataattctgaaaatagtaatattacggcacatcaaagtaaattcgttgtatatgatagtatgtttgataattttagcattaacttttgggtaaggactcctaaatataataataatgatatacaaacttatcttcaaaatgagtatacaataattagttgtataaaaaatgactcaggatggaaagtatctattaagggaaatagaataatatggacattaatagatgttaatgcaaaatctaaatcaatatttttcgaatatagtataaaagataatatatcagattatataaataaatggttttccataactattactaatgatagattaggtaacgcaaatatttatataaatggaagtttgaaaaaaagtgaaaaaattttaaacttagatagaattaattctagtaatgatatagacttcaaattaattaattgtacagatactactaaatttgtttggattaaggattttaatatttttggtagagaattaaatgctacagaagtatcttcactatattggattcaatcatctacaaatactttaaaagatttttgggggaatcctttaagatacgatacacaatactatctgtttaatcaaggtatgcaaaatatctatataaagtattttagtaaagcttctatgggggaaactgcaccacgtacaaactttaataatgcagcaataaattatcaaaatttatatcttggtttacgatttattataaaaaaagcatcaaattctcggaatataaataatgataatatagtcagagaaggagattatatatatcttaatattgataatatttctgatgaatcttacagagtatatgttttggtgaattctaaagaaattcaaactcaattatttttagcacccataaatgatgatcctacgttctatgatgtactacaaataaaaaaatattatgaaaaaacaacatataattgtcagatactttgcgaaaaagatactaaaacatttgggctgtttggaattggtaaatttgttaaagattatggatatgtttgggatacctatgataattatttttgcataagtcagtggtatctcagaagaatatctgaaaatataaataaattaaggttgggatgtaattggcaattcattcccgtggatgaaggatggacagaataa

SEQ ID NO: 14 - protein, 1297 aa, Clostridium botulinum
  misc_feature (7)..(7): Xaa can be any naturally occurring amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 15 - DNA, 4400 bp, Clostridium tetani
tagcattaaaaaaattagaacctatagtaaataaattaattaatatatagtttttataatttaattatgaataatattcttaagataaaaagtaaatttttaaaaatttaaattttcagtttacaaaaaataacctgattatgttatatgtaattgtaaaaaacatataaaaaatcagaaaaatttaggaggtatattattaatggattaaataataattttttaatttacttttgattaataaatattaaatgtttattttaattaggagatgatacgtatgccaataaccataaataattttagatatagtgatcctgttaataatgatacaattattatgatggagccaccatactgtaagggtctagatatctattataaggctttcaaaataacagatcgtatttggatagtgccggaaaggtatgaatttgggacaaaacctgaagattttaacccaccatcttcattaatagaaggtgcatctgagtattacgatccaaattatttaaggactgattctgataaagatagatttttacaaaccatggtaaaactgtttaacagaattaaaaacaatgtagcaggtgaagccttattagataagataataaatgccataccttaccttggaaattcatattccttactagacaagtttgatacaaactctaattcagtatcttttaatttattagaacaagaccccagtggagcaactacaaaatcagcaatgctgacaaatttaataatatttggacctgggcctgttttaaataaaaatgaggttagaggtattgtattgagggtagataataaaaattacttcccatgtagagatggttttggctcaataatgcaaatggcattttgcccagaatatgtacctacctttgataatgtaatagaaaatattacgtcactcactattggcaaaagcaaatattttcaagatccagcattactattaatgcacgaacttatacatgtactacatggtttatacggaatgcaggtatcaagccatgaaattattccatccaaacaagaaatttatatgcagcatacatatccaataagtgctgaagaactattcacttttggcggacaggatgctaatcttataagtattgatataaaaaacgatttatatgaaaaaactttaaatgattataaagctatagctaacaaacttagtcaagtcactagctgcaatgatcccaacattgatattgatagctacaaacaaatatatcaacaaaaatatcaattcgataaagatagcaatggacaatatattgtaaatgaggataaatttcagatactatataatagcataatgtatggttttacagagattgaattgggaaaaaaatttaatataaaaactagactttcttattttagtatgaatcatgaccctgtaaaaattccaaatttattagatgatacaatttacaatgatacagaaggatttaatatagaaagcaaagatctgaaatctgaatataaaggacaaaatatgagggtaaatacaaatgcttttagaaatgttgatggatcaggcctagtttcaaaacttattggcttatgtaaaaaaattataccaccaacaaatataagagaaaatttatataatagaactgcatcattaacagatttaggaggagaattatgtataaaaattaaaaatgaagatttaacttttatagctgaaaaaaatagcttttcagaagaaccatttcaagatgaaatagttagttataatacaaaaaataaaccattaaattttaattattcgctagataaaattattgtagattataatctacaaagtaaaattacattacctaatgataggacaaccccagttacaaaaggaattccatatgctccagaatataaaagtaatgctgcaagtacaatagaaatacataatattgatgacaatacaatatatcaatatttgtatgctcaaaaatctcctacaactctacaaagaataactatgactaattctgttgatgacgcattaataaattccaccaaaatatattcatattttccatctgtaatcagtaaagttaaccaaggtgcacaaggaattttattcttacagtgggtgagagatataattgatgattttaccaatgaatcttcacaaaaaactactattgataaaatttcagatgtatccactattgttccttatataggacccgcattaaacattgtaaaacaaggctatgagggaaactttataggcgctttagaaactaccggagtggttttattattagaatatattccagaaattactttaccagtaattgcagctttatctatagcagaaagtagcacacaaaaagaaaagataataaaaacaatagataactttttagaaaaaagatatgaaaaatggattgaagtatataaactagtaaaagcaaaatggttaggcacagttaatacgcaattccaaaaaagaagttatcaaatgtatagatctttagaatatcaagtagatgcaataaaaaaaataatagactatgaatataaaatatattcaggacctgataaggaacaaattgccgacgaaattaataatctgaaaaacaaacttgaagaaaaggctaataaagcaatgataaacataaatatatttatgagggaaagttctagatcatttttagttaatcaaatgattaacgaagctaaaaagcagttattagagtttgatactcaaagcaaaaatattttaatgcagtatataaaagcaaattctaaatttataggtataactgaactaaaaaaattagaatcaaaaataaacaaagttttttcaacaccaattccattttcttattctaaaaatctggattgttgggttgataatgaagaagatatagatgttatattaaaaaagagtacaattttaaatttagatattaataatgatattatatcagatatatctgggtttaattcatctgtaataacatatccagatgctcaattggtgcccggaataaatggcaaagcaatacatttagtaaacaatgaatcttctgaagttatagtgcataaagctatggatattgaatataatgatatgtttaataattttaccgttagcttttggttgagggttcctaaagtatctgctagtcatttagaacaatatggcacaaatgagtattcaataattagctctatgaaaaaacatagtctatcaataggatctggttggagtgtatcacttaaaggtaataacttaatatggactttaaaagattccgcgggagaagttagacaaataacttttagggatttacctgataaatttaatgcttatttagcaaataaatgggtttttataactattactaatgatagattatcttctgctaatttgtatataaatggagtacttatgggaagtgcagaaattactggtttaggagctattagagaggataataatataacattaaaactagatagatgtaataataataatcaatacgtttctattgataaatttaggatattttgcaaagcattaaatccaaaagagattgaaaaattatacacaagttatttatctataacctttttaagagacttctggggaaaccctttacgatatgatacagaatattatttaataccagtagcttctagttctaaagatgttcaattgaaaaatataacagattatatgtatttgacaaatgcgccatcgtatactaacggaaaattgaatatatattatagaaggttatataatggactaaaatttattataaaaagatatacacctaataatgaaatagattcttttgttaaatcaggtgattttattaaattatatgtatcatataacaataatgagcacattgtaggttatccgaaagatggaaatgcctttaataatcttgatagaattctaagagtaggttataatgccccaggtatccctctttataaaaaaatggaagcagtaaaattgcgtgatttaaaaacctattctgtacaacttaaattatatgatgataaaaatgcatctttaggactagtaggtacccataatggtcaaataggcaacgatccaaatagggatatattaattgcaagcaactggtactttaatcatttaaaagataaaattttaggatgtgattggtactttgtacctacagatgaaggatggacaaatgattaaacagattgatatgttcatgattactctatataaaaaattaaataatataacaatctagctatattatttttgattattttcttaatatatactaataaaataatcaaaatagagcctatcttaaattactgaagggctgtgtcaaaataagattttgacacagcctctactt

SEQ ID NO: 16 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

(19) United States
(12) Patent Application Publication
(10) Pub. No.: US 2013/0158235 A1
(43) Pub. Date: Jun. 20, 2013

(54) SELECTIVE MANUFACTURE OF RECOMBINANT NEUROTOXIN POLYPEPTIDES

(71) Applicants: Swen Grein, Rodheimer (DE); Kerstin Hölscher, Oberursel (DE); Tim Stoveken, Karben (DE); Karl-Heinz Eisele, Frankfurt Am Main (DE)

(72) Inventors: Swen Grein, Rodheimer (DE); Kerstin Hölscher, Oberursel (DE); Tim Stoveken, Karben (DE); Karl-Heinz Eisele, Frankfurt Am Main (DE)

(73) Assignee: MERZ PHARMA GmbH & CO. KGaA, FRANKFURT am MAIN (DE)

(21) Appl. No.: 13/814,633

(22) PCT Filed: Aug. 10, 2011
(86) PCT No.: PCT/EP2011/063774

§ 371 Date: Feb. 19, 2013

Related U.S. Application Data
(60) Provisional application No. 61/401,334, filed on Aug. 11, 2010.

(30) Foreign Application Priority Data
Aug. 11, 2010 (EP) 10172526.5

(51) Int. Cl. C07K 14/33

(52) U.S. Cl. CPC C07K 14/33
USPC 530/350; 536/23.4; 536/23.2; 435/320.1; 435/252.3; 435/68.1

(57) ABSTRACT

The present invention pertains to recombinant neurotoxin polypeptides and the manufacture thereof. Specifically, it relates to a polynucleotide encoding a neurotoxin polypeptide comprising a light chain, a linker and a heavy chain, wherein the linker is a modified linker comprising an heterologous amino acid sequence which confers at least one physicochemical property to the polypeptide which allows for separation of partially processed and/or unprocessed neurotoxin polypeptides from processed neurotoxin polypeptides, said heterologous amino acid sequence being flanked N- and C-terminally by a protease recognition and cleavage site Further encompassed by the present invention are vectors and host cells comprising the polynucleotide of the invention as well as polypeptides encoded by the polynucleotide and methods for the manufacture of processed neurotoxin polypeptide.

The present invention pertains to recombinant neurotoxin polypeptides and the manufacture thereof. Specifically, it relates to a polynucleotide encoding a neurotoxin polypeptide comprising a light chain, a linker and a heavy chain, wherein the linker is a modified linker comprising an heterologous amino acid sequence which confers at least one physicochemical property to the polypeptide which allows for separation of partially processed and/or unprocessed neurotoxin polypeptides from processed neurotoxin polypeptides, said heterologous amino acid sequence being flanked N- and C-terminally by a protease recognition and cleavage site. Further encompassed by the present invention are vectors and host cells comprising the polynucleotide of the invention as well as polypeptides encoded by the said polynucleotide and methods for the manufacture of processed neurotoxin polypeptide.

Clostridium botulinum and Clostridium tetani produce highly potent neurotoxins, i.e. botulinum toxins (BoNTs) and tetanus toxin (TeNT), respectively. These Clostridial neurotoxins specifically bind to neuronal cells and disrupt neurotransmitter release. Each toxin is synthesized as an inactive single-chain protein of approximately 150 kDa. The posttranslational processing involves formation of disulfide bridges, and limited proteolysis (nicking) by bacterial protease(s). Active dichain neurotoxin consists of two chains, an N-terminal light chain of approx. 50 kDa and a heavy chain of approx. 100 kDa linked by a disulfide bond. Neurotoxins structurally consist of three domains, i.e. the catalytic light chain, the heavy chain encompassing the translocation domain (N-terminal half) and the receptor binding domain (C-terminal half), see Krieglstein 1990, Eur J Biochem 188, 39; Krieglstein 1991, Eur J Biochem 202, 41; Krieglstein 1994, J Protein Chem 13, 49.

Clostridium botulinum secretes seven antigenically distinct serotypes designated A to G of the botulinum neurotoxin (BoNT). All serotypes together with the related tetanus neurotoxin (TeNT) secreted by Clostridium tetani, are Zn²⁺-endoproteases that block synaptic exocytosis by cleaving SNARE proteins. CNTs cause the flaccid muscular paralysis seen in botulism and tetanus, see Fischer 2007, PNAS 104, 10447.

Despite its toxic effects, botulinum toxin complex has been used as a therapeutic agent in a large number of diseases. Botulinum toxin serotype A was approved for human use in the United States in 1989 for the treatment of strabism, blepharospasm, and other disorders. It is commercially available as a Botulinum toxin A protein preparation, for example, under the tradename BOTOX (Allergan Inc) and under the tradename DYSPORT (Ipsen Ltd). For therapeutic application the complex is injected directly into the muscle to be treated. At physiological pH, the toxin is released from the protein complex and the desired pharmacological effect takes place. An improved BoNT/A preparation being free of complexing proteins is available under the tradename XEOMIN (Merz Pharmaceuticals GmbH). The effect of Botulinum toxin is only temporary, which is the reason why repeated administration of Botulinum toxin may be required to maintain a therapeutic effect.

The Clostridial neurotoxins weaken voluntary muscle strength and are effective in the treatment of strabism, focal dystonia, including cervical dystonia, and benign essential blepharospasm. They have been further shown to relief hemifacial spasm, and focal spasticity, and moreover, to be effective in a wide range of other indications, such as gastrointestinal disorders, hyperhidrosis, and cosmetic wrinkle correction, see Jost 2007, Drugs 67, 669.

For the manufacture of Clostridial neurotoxins, whether isolated from the originating wild-type clostridial bacteria, or from other host cell species such as E. coli, the purification of the neurotoxin from the host cell lysates after fermentation is of particular importance. In the classical production process from Clostridium botulinum, different precipitation- and extraction steps followed by a concentration step and further distinct chromatographic steps are usually applied in order to obtain purified neurotoxin, see DasGupta 1984, Toxicon 22, 415; Sathyamoorthy 1985, J Biol Chemistry 260, 10461. Similarly, in a recombinant production process from other host cells such as E. coli, various processing steps such as cell rupture, lysate clarification, and multiple chromatographic purification steps are used for isolation of the neurotoxin.

Currently, all available neurotoxin preparations comprise, in addition to the desired active (processed) dichain neurotoxin, a proteolytically unprocessed precursor and/or partially processed neurotoxin polypeptide. The proteolytically unprocessed precursor or partially processed polypeptide differs from the active (processed) dichain neurotoxin polypeptide in only a few amino acids. Therefore, they can hardly be distinguished based on their chemical and physical properties. On the other hand, the ratio of proteolytically unprocessed precursor and/or partially processed neurotoxin polypeptide of the total protein ratio is still significant in such preparations. Said amount of unprocessed precursor and/or partially processed neurotoxin is inherent to the biological production systems, which are not completely controllable. Thus, the amount of undesired proteolytically unprocessed precursor and/or partially processed Neurotoxin polypeptide in Neurotoxin preparations is predefined and, currently, rather difficult to reduce.

Processing of neurotoxins was also achieved by incubation of the precursor polypeptide with an E. coli lysate containing a protease activity elicited by an unknown E. coli protease which is capable of cleaving the neurotoxin polypeptides such that mature dichain polypeptides are obtained (see WO2006/076902).

Means and methods for a more efficient manufacture of neurotoxins by reducing the amount of the unprocessed and/or partially processed neurotoxin polypeptides and thereby improving the quality of neurotoxin preparations are highly desirable but not yet available.

Thus, the technical problem underlying the present invention may be seen as the provision of means and methods for improving the manufacture of neurotoxin polypeptides by complying with the aforementioned needs. The technical problem is solved by the embodiments characterized in the claims and herein below.

The present invention relates, thus, to a polynucleotide encoding a neurotoxin polypeptide comprising a light chain, a linker and a heavy chain, wherein the linker is a modified linker comprising an heterologous amino acid sequence which confers at least one physicochemical property to the polypeptide which allows for separation of partially processed and/or unprocessed neurotoxin polypeptides from processed neurotoxin polypeptides, said heterologous amino acid sequence being flanked N- and C-terminally by a protease recognition and cleavage site.

The term “polynucleotide” as used herein refers to single- or double-stranded DNA molecules as well as to RNA molecules. Encompassed by the said term is genomic DNA, cDNA, hnRNA, mRNA as well as all naturally occurring or artificially modified derivatives of such molecular species. The polynucleotide may be in an aspect a linear or circular molecule. Moreover, in addition to the nucleic acid sequences encoding the aforementioned neurotoxin polypeptide, a polynucleotide of the present invention may comprise additional sequences required for proper transcription and/or translation such as 5′- or 3′-UTR sequences. The polynucleotide of the present invention encodes a neurotoxin polypeptide comprising a modified linker. The neurotoxin polypeptide and, in particular, its light chain and heavy chain are derivable from one of the antigenically different serotypes of Botulinum Neurotoxins, i.e. BoNT/A, BoNT/B, BoNT/C1, BoNT/D, BoNT/E, BoNT/F, BoNT/G, or Tetanus Neurotoxin (TeNT). In an aspect, said light and heavy chain of the neurotoxin polypeptide are the light and heavy chain of a neurotoxin selected from the group consisting of: BoNT/A, BoNT/B, BoNT/C1, BoNT/D, BoNT/E, BoNT/F, BoNT/G or TeNT. In another aspect, the said polynucleotide comprises a nucleic acid sequence as shown in SEQ ID NO: 1 (BoNT/A), SEQ ID NO: 3 (BoNT/B), SEQ ID NO: 5 (BoNT/C1), SEQ ID NO: 7 (BoNT/D), SEQ ID NO: 9 (BoNT/E), SEQ ID NO: 11 (BoNT/F), SEQ ID NO: 13 (BoNT/G) or SEQ ID NO: 15 (TeNT) which has been modified in the linker as specified herein. Moreover, encompassed is in an aspect a polynucleotide comprising a nucleic acid sequence encoding an amino acid sequence as shown in any one of SEQ ID NO: 2 (BoNT/A), SEQ ID NO: 4 (BoNT/B), SEQ ID NO: 6 (BoNT/C1), SEQ ID NO: 8 (BoNT/D), SEQ ID NO: 10 (BoNT/E), SEQ ID NO: 12 (BoNT/F), SEQ ID NO: 14 (BoNT/G) or SEQ ID NO: 16 (TeNT) which has been modified in the linker as specified herein.

In another aspect, the said polynucleotide is a variant of the aforementioned polynucleotides comprising one or more nucleotide substitutions, deletions and/or additions which in still another aspect may result in a polypeptide having one or more amino acid substitutions, deletions and/or additions. Moreover, a variant polynucleotide of the invention shall in another aspect comprise a nucleic acid sequence variant being at least 40%, at least 50%, at least 60%, at least 70%, at least 75%, at least 80%, at least 85%, at least 90%, at least 95%, at least 98% or at least 99% identical to the nucleic acid sequence as shown in any one of SEQ ID NOs: 1, 3, 5, 7, 9, 11, 13 or 15 or a nucleic acid sequence variant which encodes an amino acid sequence being at least 40%, at least 50%, at least 60%, at least 70%, at least 75%, at least 80%, at least 85%, at least 90%, at least 95%, at least 98% or at least 99% identical to the amino acid sequence as shown in any one of SEQ ID NOs: 2, 4, 6, 8, 10, 12, 14, or 16. The term “identical” as used herein refers to sequence identity characterized by determining the number of identical amino acids between two nucleic acid sequences or amino acid sequences wherein the sequences are aligned so that the highest order match is obtained. It can be calculated using published techniques or methods codified in computer programs such as, for example, BLASTP, BLASTN or FASTA (Altschul 1990, J Mol Biol 215, 403). The percent identity values are, in one aspect, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (Higgins 1989, CABIOS 5, 151) or the programs Gap and BestFit (Needleman 1970, J Mol Biol 48; 443; Smith 1981, Adv Appl Math 2, 482), which are part of the GCG software packet (Genetics Computer Group 1991, 575 Science Drive, Madison, Wis., USA 53711), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an aspect, each of the aforementioned variant polynucleotides encodes a polypeptide retaining one or more and, in another aspect, all of the biological properties of the respective Neurotoxin polypeptide, i.e. the BoNT/A, BoNT/B, BoNT/C1, BoNT/D, BoNT/E, BoNT/F, BoNT/G or Tetanus Neurotoxin (TeNT). Those of skill in the art will appreciate that full biological activity is maintained only after proteolytic activation, even though it is conceivable that the unprocessed precursor can exert some biological functions or be partially active. “Biological properties” as used herein refers to (a) receptor binding, (b) internalization, (c) translocation across the endosomal membrane into the cytosol, and/or (d) endoproteolytic cleavage of proteins involved in synaptic vesicle membrane fusion. In vivo assays for assessing biological activity include the mouse LD50 assay and the ex vivo mouse hemidiaphragm assay as described by Pearce et al. (Pearce 1994, Toxicol Appl Pharmacol 128: 69-77) and Dressler et al. (Dressler 2005, Mov Disord 20:1617-1619, Keller 2006, Neuroscience 139: 629-637). The biological activity is commonly expressed in Mouse Units (MU). As used herein, 1 MU is the amount of neurotoxic component, which kills 50% of a specified mouse population after intraperitoneal injection, i.e. the mouse i.p. LD50. In a further aspect, the variant polynucleotides can encode Neurotoxins having improved or altered biological properties, e.g., they may comprise cleavage sites which are improved for enzyme recognition or may be improved for receptor binding or any other property specified above.

Neurotoxin polypeptides as referred to herein comprise an N-terminal light chain, an intermediate linker and a C-terminal heavy chain. The neurotoxins are translated as single chain precursor molecules and become proteolytically cleaved into a mature dichain form during processing. Proteolytical cleavage occurs at the linker in a manner such that the linker is either cleaved once or is removed after cleavage at the N- and C-termini of the linker. Dependent on the efficacy of the processing, in addition to the uncleaved single chain construct, partially processed neurotoxin polypeptides may also be generated, i.e. neurotoxin polypeptides which retain the linker either at the light or heavy chain due to improper cleavage. The endogenous linker of the neurotoxin polypeptides is modified in accordance with the present invention such that it comprises a heterologous (i.e. not endogenous) amino acid sequence which binds with high affinity to a purification matrix, said heterologous amino acid sequence being flanked N- and C-terminally by a protease recognition and cleavage site. Said modification, in an aspect, may be a modification of individual amino acids in the endogenous linker yielding the modified linker. Alternatively and in another aspect, said modification may be the replacement of the endogenous linker by a heterologous linker. Suitable techniques for carrying out such modifications to the linker are well known in the art and include mutagenesis techniques as well as standard cloning and PCR based techniques.

The term “heterologous amino acid sequence which confers at least one physicochemical property to the polypeptide which allows for separation of partially processed or unprocessed neurotoxin polypeptides from processed neurotoxin polypeptides” refers to an amino acid sequence which does not naturally occur in the linker (i.e. heterologous) of the neurotoxin to be modified and which confers at least one physicochemical property to the neurotoxin molecular species comprising the said linker, i.e. the unprocessed and/or partially processed neurotoxin. The said physicochemical property, in an aspect, shall be an altered affinity for a purification matrix as defined elsewhere herein in detail or may be an altered physical property such as the molecular weight which allows for efficient physical separation.

In an aspect of the polynucleotide of the present invention, said heterologous amino acid sequence is a heterologous amino acid sequence which binds with high affinity to a purification matrix.

The term “heterologous amino acid sequence which binds with high affinity to a purification matrix” relates to an amino acid sequence which does not endogenously (i.e. naturally occur) in the linker. Said amino acid sequence shall confer high affinity binding to a purification matrix. Dependent on the purification matrix to be used, this can be achieved either by individual amino acids which are present in the sequence and which are capable of physically or chemically interacting with matrix components or peptide sequence stretches or three-dimensional structures formed thereby being capable of interacting with the matrix. The latter case of high affinity binding includes also binding based on protein-protein interactions. Suitable high affinity protein-protein interactions which can be applied in this context are antibody-epitope interactions. For example, the purification matrix may contain an antibody which specifically recognizes and binds with high affinity to a epitope comprised by the heterologous amino acid sequence. Further, receptor-ligand or other hight affinity protein-protein interactions can be applied. For example, the purification matrix may be coupled to biotin and the heterologous amino acid sequence may comprise streptavidin or avidin or tags derived therefrom. Further heterologous amino acid sequences to be used in particular aspects of the present invention are specified elsewhere herein. A “purification matrix” as meant herein refers to a three dimensional structure or spatial arrangement capable of binding to the aforementioned amino acid sequence of the modified linker. Well-known matrices comprise polypeptides, glass, polystyrene, polypropylene, polyethylene, polyethylene glycol (PEG), dextran, nylon, amylases, natural and modified celluloses, polyacrylamides, gabbros, and magnetite. A solid matrix is, in an aspect of the invention, a polysaccharide matrix selected from the group consisting of: sepharose, sephadex, superdex; agarose, sephacell, micro-cellulose, and alginate-beads. In another aspect, said solid matrix can consist of glass-beads, and/or polypeptide matrices.

In a further aspect of the polynucleotide of the invention, said heterologous amino acid sequence increases the molecular weight of the neurotoxin polypeptide such that the partially processed and/or unprocessed neurotoxin polypeptides can be physically separated from processed neurotoxin polypeptide.

The said heterologous amino acid sequence which is comprised in this aspect by the linker will increase the molecular weight of the linker containing neurotoxin molecular species such that the said linker containing molecular species can be separated from the processed neurotoxin, i.e. the molecular species which does not contain the modified linker, by physical separation processes. The said processes, in an aspect, include size exclusion chromatography, ultrafiltration, preparative gel electrophoresis technologies, or centrifugation based technologies. Amino acids which can be introduced into the modified linker to increase to molecular weight include essentially all available naturally occurring or artificial amino acids which are capable of being introduced via peptide bonds into the peptide backbone. Moreover, in an aspect the amino acids may be chemically modified in order to improve or facilitate the physical separation. In such a case, moieties which contribute a high molecular weight can be coupled to the amino acids. In another aspect, a larger stretch of unmodified amino acids may be sufficient to increase the molecular weight of the modified linker to an extent which allows for physical separation.

It will be understood that in an aspect, the modified linker comprises (i) a heterologous amino acid sequence which binds with high affinity to a purification matrix and (ii) a heterologous amino acid sequence which increases the molecular weight of the neurotoxin polypeptide such that the partially processed and/or unprocessed neurotoxin polypeptides can be physically separated from processed neurotoxin polypeptide.

The said heterologous amino acid sequence shall be N- and C-terminally flanked by a protease recognition and cleavage site. It is envisaged by the present invention that the linker will be entirely removed after the processing is completed. This can be achieved by introducing two protease recognition and cleavage sites flanking the N- and C-terminal end of the linker sequence. Upon cleavage of the cleavage sites, a peptide with the linker sequence will be released and the dimerized light and heavy chain of the neurotoxin polypeptide remain as the mature dichain neurotoxin polypeptide. In an aspect, it is envisaged to introduce at the N- and C-terminal end of the linker sequence recognition and cleavage sites for the same protease. In another aspect, it is envisaged to introduce at the N- and C-terminal end of the linker sequence recognition and cleavage sites for different proteases. Dependent on the protease to be used in accordance with the present invention, the protease recognition site may differ from the cleavage site or may be identical thereto. Moreover, an exogenous protease can be used in an aspect according to the present invention. Alternatively, an endogenous protease activity which is present in the linker or other parts of the neurotoxin polypeptide, such as the neurotoxin protease activity, encoded by the polynucleotide of the invention can be used in another aspect of the invention. The latter autocatalytic protease cleavage system for a single cleavage site has been described in more detail in U.S. Pat. No. 7,556,817 which is herewith incorporated by reference. In an aspect, said protease recognition and cleavage site is selected from the group consisting of: neurotoxin light chain protease recognition and cleavage site from SNAP 25, recognition and cleavage site from an E. coli protease, Thrombin recognition and cleavage site, Factor X recognition and cleavage site, Trypsin recognition and cleavage site, Enterokinase recognition and cleavage site, TEV protease recognition and cleavage site, HRV 3c recognition and cleavage site, and PreScission Protease recognition and cleavage site.

Advantageously, it has been found in accordance with the present invention that a neurotoxin polypeptide with a modified linker as specified above can be more efficiently purified due to the presence of heterologous amino acid sequences conferring altered physicochemical properties to linker containing polypeptides which allow for an efficient and reliable purification, such as affinity binding to a matrix or increased molecular weight which allows for improved physical separation. Moreover, solubility can be increased dependent on the size and/or the hydrophilicity of the linker. In addition, further properties which are suitable for the manufacture of neurotoxin polypeptides can be introduced into the linker, such as detectable markers, e.g., fluorescent proteins. These properties persist only during processing since the respective amino acid sequence will be removed as a result of the processing steps from the mature neurotoxin polypeptides.

In an aspect of the polynucleotide of the present invention, said heterologous amino acid sequence which binds with high affinity to a purification matrix comprises a purification tag.

The term “purification tag” refers to an amino acid sequence which allows for purification of the polynucleotide comprising it. This can be achieved in an aspect due to the presence of individual amino acids, peptide sequences or three dimensional peptide structures which are capable of physically or chemically interacting with the purification matrix. Suitable tags include affinity tags and epitope tags. In this context, it is well known that certain amino acids are capable of forming complexes with defined matrices to be used for purification, such as histidines which are capable of physico-chemically interacting with metal ions such as nickel or cobalt embedded in a suitable matrix such as sepharose or agarose. Further peptide sequences or three dimensional structures can be found in chitin binding proteins (CBP), maltose binding proteins (MBP) or glutathione-S-transferases (GST). In a further aspect, protein-protein interaction based purification matrix/purification tag systems can be used such as streptavidin/biotin systems which are also well known in the art. In yet another aspect, the tag may confer a physic-chemical property to the polypeptide containing it which allows for purification, such as fluorescence. Suitable tags in this context include the well known fluorescent proteins, such as green fluorescent protein (GFP), blue fluorescent protein (BFP) or others. In addition to these tags which have an inherent affinity for some matrices, epitope tags can be applied in another aspect. Such epitope tags consists of short amino acid stretches which constitute a peptide epitope that can be specifically recognized by an antibody. Epitope tags are also well known in the art and include FLAG tags, MYC-tags, or HA-tags. Thus, in an aspect, the purification tag is selected from the group consisting of: His-tag, Myc-tag, FLAG-tag, strep-tag, MBP-tag, NusA tag, GST-tag, streptavidin and avidin.

In another aspect of the polynucleotide of the invention, said heterologous amino acid sequence which binds with high affinity to a purification matrix comprises (i) at least one amino acid domain conferring high affinity binding to the purification matrix and (ii) at least one amino acid domain which prevents interference of the heterologous amino acid sequence with the formation of proper disulfide bonds between the light and heavy chain.

In an aspect, said at least one domain conferring high affinity binding to the purification matrix comprises a purification tag as described elsewhere herein. In a further aspect, said at least one amino acid domain enables and/or facilitates proper disulfide bond formation between the heavy and the light chain and/or prevents interference of the heterologous amino acid sequence with the formation of proper disulfide bonds between the light and heavy chain. In an aspect, the said domain forms a three dimensional structure wherein the C- and N-terminal amino acids of the heterologous amino acid sequence are in physical proximity. As a result of the physical proximity of the N- and C-terminal amino acid which, in an aspect, is in the range of the proximity of the sulphur atoms of a disulphide bound, i.e. about 2.0 Angstrom, the remaining part of the linker will protrude from the light and heavy chain of the neurotoxin molecule. Accordingly, the properties which are conferred by the linker such as high affinity binding or an increased solubility of the molecule, are not in physical proximity and, thus, do not interfere with proper intramolecular disulphide bond formation between the light and the heavy chain. Moreover, in an aspect the protruding linker also prevents sterically the formation of intermolecular disulphide bond formation between separate neurotoxin molecules. In an aspect, the said three dimensional structure, which brings the cysteine residues forming the intramolecular disulphide bridge between light and heavy chain in close proximity, is an antiparallel coiled coil or an antiparallel beta-sheet structure. In an aspect, said antiparallel coiled coil is derived from 1FHA ferritin, 1VSG variant surface glycoprotein, 1LIGb Asp receptor, 1PRCm photosynthetic reaction centre, 2CCY cytochrome C′, 256B cyctochrome b562, 3LZM T4 lysozyme, 1COL1 a colicin, 1PRC1photosynthetic reaction centre, 2CTS citrate synthase, 1ROP Rop protein, 3HHR human growth hormone or 1LPE apolipoprotein A3. How to generate such structures is well known in the art and described, e.g., in Gernert 1995, Protein Science 4(11): 2252-22560 or Hadley 2008, Proc Natl. Acad. Sci. USA 15:105(2):530-5, both are herewith incorporated by reference.

In another aspect of the polynucleotide of the invention, said heterologous amino acid sequence further comprises at least one detectable marker amino acid sequence.

In an aspect, said detectable marker amino acid sequence is selected from the group consisting of: an amino acid sequence of a fluorescent protein, an amino acid sequence of an enzyme capable of generating a detectable signal, and an amino acid sequence of a detectable tag. In an aspect, the fluorescent protein is GFP or BFP. In another aspect, the enzyme capable of generating a detectable signal is selected from beta galactosidase (GAL4), firefly luciferase, or chloramphenicol transferase (CAT). In yet another aspect, the detectable tag is an epitope tag such as those specified elsewhere in this specification (NusA-Tag).

In another aspect, the modified linker comprises further nucleic acid sequences which encode polypeptide domains conferring properties to the polypeptide which improve the purification or enable or improve solubility. Such polypeptide domains, in an aspect, may be additional purification tags as recited elsewhere herein and which allow for an improved purification via affinity binding. Further, polypeptide domains may enable or facilitate solubility due to the presence of hydrophilic amino acid residues. Suitable domains are well known in the art.

It is to be understood that the definitions and explanations of the terms made above apply mutatis mutandis for all aspects described in this specification in the following except as otherwise indicated.

The present invention also relates to a vector comprising the polynucleotide of the present invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site- directed integration of the targeting construct into genomic DNA. Such target constructs, preferably, comprise DNA of sufficient length for either homologous or heterologous recombination as described in detail below. The vector encompassing the polynucleotides of the present invention, in an aspect, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. Moreover, in an aspect of the invention, the polynucleotide is operatively linked to expression control sequences allowing expression in prokaryotic or eukaryotic host cells or isolated fractions thereof in the said vector. Expression of the polynucleotide comprises transcription of the polynucleotide into a translatable mRNA. Regulatory elements ensuring expression in host cells are well known in the art. In an aspect, they comprise regulatory sequences ensuring initiation of transcription and/or poly-A signals ensuring termination of transcription and stabilization of the transcript. Additional regulatory elements may include transcriptional as well as translational enhancers. Possible regulatory elements permitting expression in prokaryotic host cells comprise, e.g., the lac-, trp- or tac-promoter in E. coli, and examples for regulatory elements permitting expression in eukaryotic host cells are the AOX1- or the GAL1-promoter in yeast or the CMV-, SV40-, RSV-promoter (Rous sarcoma virus), CMV-enhancer, SV40-enhancer or a globin intron in mammalian and other animal cells. Other expression systems envisaged by the invention shall permit expression in insect cells, such as polyhedrin promoter based systems.

Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (Invitrogen) or pSPORT1 (Invitrogen) or baculovirus-derived vectors. Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The present invention further pertains to a host cell comprising the polynucleotide or the vector of the present invention.

The term “host cell” as used herein encompasses prokaryotic and eukaryotic host cells. In an aspect the host cell is a bacterial cell and, in another aspect, a Firmicutes bacterial cell. In one aspect, the said bacterial host cell is an E. coli host cell. In another aspect, it is a Clostridium host cell. In a further aspect, the said Clostridium host cell is a Clostridium botulinum host cell, in even a further aspect, a cell of one of the aforementioned seven different serotypes of Clostridium botulinum. In yet another aspect, the bacterial host cell is a Clostridium tetani host cell. In a further aspect, the host cell is a Bacillus host cell and in a particular aspect a Bacillus megaterium host cell. A eukaryotic host cell, in an aspect, is a cell of an animal cell line suitable for production of toxic proteins or a fungal host cell such as a yeast host cell. A host cell as referred to herein, thus, encompasses in an aspect yeast, mammalian, plant or insect cells either as primary cells or as cell lines.

The present invention also relates to a polypeptide encoded by the polynucleotide of the present invention.

The term “polypeptide” as used herein encompasses isolated or essentially purified polypeptides being essentially free of other polypeptides including the complexing proteins (HA70, HA17, HA33, or NTNH (NBP)) of the host cell or polypeptide preparations comprising other proteins in addition. Moreover, the term includes chemically modified polypeptides. Such modifications may be artificial modifications or naturally occurring modifications. As referred to above, the polypeptide of the present invention shall have the biological properties of the Neurotoxin polypeptides referred to above. The polypeptide of the invention, in an aspect, can be manufactured by a method of manufacturing a polypeptide as described elsewhere herein in more detail. In an aspect of the invention, a polypeptide preparation is also envisaged which comprises a complex of the Neurotoxin polypeptide and its complexing proteins referred to above.

Moreover, the present invention encompasses a method for the manufacture of a processed neurotoxin polypeptide comprising the steps of:
a) contacting the unprocessed neurotoxin polypeptide of the present invention with a purification matrix under conditions and for a time sufficient for allowing binding of the said unprocessed neurotoxin polypeptide to the purification matrix;
b) contacting the unprocessed neurotoxin polypeptide bound to the purification matrix with a protease which is capable of cleaving the neurotoxin polypeptide at the protease recognition and cleavage sites resulting in processed and partially processed and/or unprocessed neurotoxin polypeptides; and
c) removing the released, processed neurotoxin polypeptide from the purification matrix.

It will be understood that the methods for manufacturing neurotoxin polypeptides specified herein may comprise additional steps such as steps pertaining to the synthesis of the neurotoxins, washing steps or steps concerning the formulation of the purified neurotoxin polypeptides into neurotoxin preparations which can be applied as pharmaceutical or cosmetic compositions, respectively.

The term “contacting” as used herein refers to bringing at least two different compounds in physical proximity as to allow physical and/or chemical interaction of said compounds. In the aforementioned method, unprocessed neurotoxin according to the present invention, i.e. neurotoxin polypeptides comprising a modified linker as specified above, is contacted with a purification matrix for a time and under conditions sufficient to allow binding of said unprocessed neurotoxin to the matrix. The said time and conditions will dependent on the type of binding which shall be achieved between the linker and the matrix. The person skilled in the art is well aware of which conditions need to be applied for a given combination of purification matrix and linker. Contacting may be carried out by means of chromatography or batch-wise. Upon binding of the unprocessed neurotoxin, washing steps may be performed in order to remove (recover) unbound unprocessed neurotoxin and other host cell proteins.

Subsequently, the unprocessed neurotoxin polypeptide bound to the purification matrix shall be contacted with a protease which is capable of cleaving the neurotoxin polypeptide at the protease recognition and cleavage sites under time and conditions which allow for protease activity. The protease in this context may be provided as a purified enzyme preparation or as a cell lysate from a cell expressing said protease endogenously or exogenously. As a result of the protease activity, the neurotoxin polypeptides with the modified linker will be cleaved into processed and partially processed neurotoxin polypeptides. Some neurotoxin polypeptides may even remain in uncleaved form as unprocessed neurotoxin polypeptides. Suitable conditions for the aforementioned protease treatment depend on the protease to be used and can be adapted by the skilled artisan without further ado.

The processed neurotoxin polypeptides will upon cleavage loose their modified linkers which are responsible for binding to the purification matrix and are, thus, released from the matrix. Contrary, the partially processed or unprocessed neurotoxin polypeptides remain bound to the purification matrix due to the presence of the modified linkers in said molecules. The mature, processed neurotoxin polypeptides can be removed from the purification matrix by washing with an appropriate buffer or water.

In a particular aspect of the aforementioned method, the linker of the neurotoxin polypeptide comprises an epitope tag as specified elsewhere herein and the purification matrix contains an antibody which specifically binds to the said epitope tag. In an aspect, said purification matrix is applied for column affinity chromatography as follows. A solution comprising the unprocessed neurotoxin polypeptide according to the invention is applied to the column. The solution is maintained in the column for a time and under conditions which allow specific binding of the epitope tags in the modified linkers of the unprocessed neurotoxin polypeptides to the purification matrix. Subsequently, excessive unprocessed neurotoxin polypeptide is removed by washing steps. After said washing steps, a solution comprising a protease capable of recognizing and cleaving the recognition and cleavage sites flanking the modified linker is applied. The unprocessed neurotoxin polypeptide bound to the purification matrix is incubated with the protease as to allow proteolytic cleavage at both cleavage sites. As a consequence of the said cleavage reaction, mature, processed neurotoxin polypeptide is released from the purification matrix while unprocessed or partially processed neurotoxin polypeptide remains bound to the purification matrix due to its association with the linker. The released processed neurotoxin can then be eluted from the column.

In another particular aspect of the aforementioned method, the linker of the neurotoxin polypeptide comprises a poly-histidine tag, such as a hexa-His tag, as specified elsewhere herein and the purification matrix represents a nickel-, cobalt-, copper- and zinc-containing sepharose or other chromatography media. In an aspect, said purification matrix is applied for column affinity chromatography as follows. A solution comprising the unprocessed neurotoxin polypeptide according to the invention is applied to the column. The solution is maintained in the column for a time and under conditions which allow specific binding of the hexa-His tag in the modified linkers of the unprocessed neurotoxin polypeptides to the nickel or cobalt ions in the purification matrix. Subsequently, excessive unprocessed neurotoxin polypeptide and especially contaminating host cell proteins are removed by washing steps. After said washing steps, a solution comprising a protease capable of recognizing and cleaving the recognition and cleavage sites flanking the modified linker is applied. The unprocessed neurotoxin polypeptide bound to the purification matrix is incubated with the protease as to allow proteolytic cleavage at both cleavage sites. As a consequence of the said cleavage reaction, mature, processed neurotoxin polypeptide is released from the purification matrix while unprocessed or partially processed neurotoxin polypeptide remains bound to the purification matrix due to its association with the linker. The released processed neurotoxin can than be eluted from the column.

Further, the present invention pertains to a method for the manufacture of a processed neurotoxin polypeptide comprising the steps of:
a) contacting a neurotoxin polypeptide of the present invention with a protease which is capable of cleaving the neurotoxin polypeptide at the protease recognition and cleavage sites;
b) contacting the processed, unprocessed and/or partially processed neurotoxin polypeptide obtained in step a) with a purification matrix under conditions and for a time sufficient for allowing binding of the said unprocessed and/or partially processed neurotoxin polypeptide to the purification matrix; and
c) removing the released, processed neurotoxin polypeptide from the purification matrix.

In an aspect of the aforementioned method, step a) is carried out in a host cell expressing the polynucleotide of the invention and a protease which is capable of cleaving the neurotoxin polypeptide at the protease recognition and cleavage sites. In an aspect, said protease can be expressed endogenously in the said host cell. In another aspect, the said protease is expressed from an expression construct which has been introduced into the said host cell. In an aspect, the host cell is a host cell lysate or a host cell as referred to herein elsewhere.

Moreover, the invention contemplates a method for the manufacture of a processed neurotoxin polypeptide comprising the steps of:
a) contacting a neurotoxin polypeptide of the present invention with a protease which is capable of cleaving the neurotoxin polypeptide at the protease recognition and cleavage sites;
b) subjecting the processed, unprocessed and/or partially processed neurotoxin polypeptide obtained in step a) to physical conditions for a time sufficient for allowing physical separation of partially processed or unprocessed neurotoxin polypeptides from processed neurotoxin polypeptides; and
c) removing processed neurotoxin polypeptide after said physical separation.

The term “subjecting” as used herein refers to applying the physical conditions to the processed, unprocessed and/or partially processed neurotoxin polypeptide. Dependent on the physical conditions which are applied for separation, the person skilled in the art knows how said subjecting may be carried out. In an aspect, subjecting comprises performing size exclusion chromatography, the principles of which are further defined elsewhere herein. In another aspect, subjecting comprises filtration, e.g., ultrafiltration. In another aspect, subjecting comprises centrifugation, e.g., ultracentrifugation. In yet another aspect, subjecting comprises gel electrophoresis, in an aspect, in a preparative scale. After the molecular species of the neurotoxin present after cleavage have been physically separated into the fully processed neurotoxin polypeptide molecules and the unprocessed and/or partially processed neurotoxin molecules, the said processed neurotoxin polypeptides can be removed by suitable measures well known in the art. If in an aspect size exclusion chromatography is applied for physical separation, the processed neurotoxin polypeptides can be obtained from a size exclusion chromatography column by collecting the eluate into different fractions. The processed and the unprocessed and/or partially processed neurotoxin polypeptides will be present in different fractions in such a case.

In another aspect of the methods of the invention, the manufacture of the neurotoxin polypeptide comprises further purification steps. In one aspect, said further purification steps comprise size exclusion chromatography.

By size exclusion chromatography as used in the present invention, particles are separated based on their size, i.e. on their hydrodynamic volume. A mobile phase is either an aqueous solution used to transport the sample (gel filtration chromatography), or an organic solvent (gel permeation chromatography). A stationary phase is either a gel medium (polyacrylamide, dextran or agarose) and filter under low pressure, or a silica, or crosslinked polystyrene medium under a higher pressure. In even another aspect, said size exclusion chromatography is performed as column chromatography. In a further aspect of the method of the present invention, said size exclusion chromatography is performed using molecular sieves with distinct pore sizes such as activated carbon, silica gel, zeolite.

The method of the present invention, in another aspect, further comprises ion exchange chromatography.

Ion exchange chromatography as used in the present invention separates molecules based on differences between the overall charge of the proteins and related compounds. It is used for protein purification, for purification of oligonucleotides, peptides, or other charged molecules. Such molecules may be present in the solution to be applied to the method of the purification as contaminations. The protein or the related compound of interest, in the present case the Neurotoxin, must have a charge opposite to that of the functional group attached to the resin in order to bind. Because this interaction is ionic, binding must take place under low ionic conditions. Elution is achieved by increasing the ionic strength to break up the ionic interaction, or by changing the pH of the protein. In an aspect of the method of the invention, said exchange chromatography is performed as column chromatography. The ion exchange chromatography as used in the present invention is in a further aspect performed by cation and/or anion chromatography. In anion exchange chromatography as used herein the surface charge of the solutes (proteins, peptides, nucleic acids, endotoxins) which bind will be net negative, thus to get binding of a specific protein one should be near or above the pI of that protein. Commonly used anion exchange resins are Q-resin (Q Sepharose), a Quaternary amine; and DEAE (DiEthylAminoEthane) resin. Generally, an ion exchange resin is an insoluble matrix of small beads having a charged surface, used as an artificial zeolite. Different types of resins can be distinguished based on their functional groups including strongly acidic resins (sulfonic acid groups, eg. sodium polystyrene sulfonate or polyAMPS), strongly basic resins, (quaternary amino groups, e.g. trimethylammonium groups, eg. polyAPTAC), weakly acidic resins (mostly, carboxylic acid groups), weakly basic resins (primary, secondary, and/or ternary amino groups, e.g., polyethylene amine). There are also specialised types of resins can be further distinguishes including chelating resins (iminodiacetic acid, thiourea). In cation exchange chromatography as used herein, the surface charge of the solutes (proteins, peptides, nucleic acids, endotoxins) which bind will be net positive, thus to get binding of a specific protein one should be near or below the pI of that protein. Commonly used cation exchange resins are S-resin, sulfate derivatives; and CM resins, carboxylate derived ions.

Further purification techniques which can be used in additional purification steps according to the method of the present invention include hydrophobic interaction chromatography, multimodal chromatography (e.g. Capto MMC or Capto Adhere), affinity chromatography (e.g. Blue Sepharose) and/or hydroxyapatite matrices.

In an aspect of the method of the present invention said ion exchange chromatography is carried out prior to and/or after contacting the processed, unprocessed and/or partially processed neurotoxin polypeptide with the purification matrix. In another aspect of the method of the invention, said ion exchange chromatography as used herein is carried out prior to contacting the processed, unprocessed and/or partially processed neurotoxin polypeptide with the purification matrix. Due to this measure, the risk of potential cross-reactivity or unspecific binding during affinity chromatography can be further avoided and reduced.

The method of the present invention allows for the manufacture of active processed neurotoxin polypeptide essentially free of unprocessed or partially processed precursor polypeptide and, thus, obtaining higher amounts of the active processed neurotoxin polypeptide. Such neurotoxin polypeptides can be used for formulating medicaments or cosmetic compositions. Essentially free in this context means that the unprocessed and/or partially processed neurotoxin is below a certain threshold, in an aspect, less than 2.5%, less than 1%, less than 0.5% or less than 0.1% of the total neurotoxin comprised by a given sample.

Thus, the present invention also relates to a method for the manufacture of a medicament comprising the steps of the aforementioned method and the further step of formulating the proteolytically processed neurotoxin polypeptide as medicament.

The term “medicament” as used herein refers, in one aspect, to a pharmaceutical composition containing the biologically active (proteolytically processed) neurotoxin polypeptide as pharmaceutical active compound, wherein the pharmaceutical composition may be used for human or non-human therapy of various diseases or disorders in a therapeutically effective dose.

A pharmaceutical composition as used herein comprises the biologically active (proteolytically processed) Neurotoxin polypeptide of the present invention, and in one aspect, one or more pharmaceutically acceptable carrier. The active Neurotoxin can be present in liquid or lyophilized form. In an aspect, said compound can be present together with glycerol, protein stabilizers (e.g., human serum albumin (HAS)) or non-protein stabilizers.

The pharmaceutical composition is, in one aspect, administered topically. Conventionally used drug administration is administered intra-muscular, subcutaneous (near glands). However, depending on the nature and the mode of action of a compound the pharmaceutical composition may be administered by other routes as well.

The compound, i.e. the biologically active (proteolytically processed) neurotoxin polypeptide is the active ingredient of the composition, and is in one aspect administered in conventional dosage forms prepared by combining the drug with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables.

The carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like.

A therapeutically effective dose refers to an amount of the compound to be used in a pharmaceutical composition of the present invention which prevents, ameliorates or treats the symptoms accompanying a disease or condition referred to in this specification. Therapeutic efficacy and toxicity of the compound can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

The pharmaceutical compositions and formulations referred to herein are administered at least once in order to treat or ameliorate or prevent a disease or condition recited in this specification. However, the said pharmaceutical compositions may be administered more than one time.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent. The resulting formulations are to be adapted to the mode of administration. Dosage recommendations shall be indicated in the prescribers or users instructions in order to anticipate dose adjustments depending on the considered recipient.

The medicament according to the present invention may in a further aspect of the invention comprise drugs in addition to the biologically active (proteolytically processed) neurotoxin polypeptide which are added to the pharmaceutical composition during its formulation. Finally, it is to be understood that the formulation of a pharmaceutical composition takes place under GMP standardized conditions or the like in order to ensure quality, pharmaceutical security, and effectiveness of the medicament.

The present invention, in general, contemplates a composition comprising proteolytically processed neurotoxin polypeptide obtainable by the method of the present invention.

The term “composition” refers to any composition formulated in solid, liquid, aerosol (or gaseous) form. Said composition comprises the compound of the invention optionally together with suitable auxiliary compounds such as diluents or carriers or further ingredients. In this context, it is distinguished for the present invention between auxiliary compounds, i.e. compounds which do not contribute to the effects elicited by the compound of the present invention upon application of the composition for its desired purpose, and further ingredients, i.e. compounds which contribute a further effect or modulate the effect of the compound of the present invention. Suitable diluents and/or carriers depend on the purpose for which the composition is to be used and the other ingredients. The person skilled in the art can determine such suitable diluents and/or carriers without further ado. Examples of suitable carriers and/or diluents are disclosed elsewhere herein.

In a further aspect of the invention, the aforementioned composition is a medicament as specified elsewhere in the description in more detail. In one aspect the said medicament can be used for prevention and/or treatment of at least one of the following diseases and disorders: voluntary muscle strength, focal dystonia, including cervical, cranial dystonia, and benign essential blepharospasm, hemifacial spasm, and focal spasticity, gastrointestinal disorders, hyperhidrosis, and cosmetic wrinkle correction, in a further aspect also Blepharospasm, oromandibular dystonia, jaw opening type, jaw closing type, bruxism, Meige syndrome, lingual dystonia, apraxia of eyelid, opening cervical dystonia, antecollis, retrocollis, laterocollis, torticollis, pharyngeal dystonia, laryngeal dystonia, spasmodic dysphonia/adductor type, spasmodic dysphonia/abductor type, spasmodic dyspnea, limb dystonia, arm dystonia, task specific dystonia, writer's cramp, musician's cramps, golfer's cramp, leg dystonia, thigh adduction, thigh abduction knee flexion, knee extension, ankle flexion, ankle extension, equinovarus, deformity foot dystonia, striatal toe, toe flexion, toe extension, axial dystonia, pisa syndrome, belly dancer dystonia, segmental dystonia, hemidystonia, generalised dystonia, dystonia in lubag, dystonia in corticobasal degeneration, dystonia in lubag, tardive dystonia, dystonia in spinocerebellar ataxia, dystonia in Parkinson's disease, dystonia in Huntington's disease, dystonia in Hallervorden Spatz disease, dopa-induced dyskinesias/dopa-induced dystonia, tardive dyskinesias/tardive dystonia, paroxysmal dyskinesias/dystonias, kinesiogenic non-kinesiogenic action-induced palatal myoclonus, myoclonus myokymia, rigidity, benign muscle cramps, hereditary chin trembling, paradoxic jaw muscle activity, hemimasticatory spasms, hypertrophic branchial myopathy, maseteric hypertrophy, tibialis anterior hypertrophy, nystagmus, oscillopsia supranuclear gaze palsy, epilepsia, partialis continua, planning of spasmodic torticollis operation, abductor vocal cord paralysis, recalcitant mutational dysphonia, upper oesophageal sphincter dysfunction, vocal fold granuloma, stuttering Gilles de Ia Tourette syndrome, middle ear myoclonus, protective larynx closure, postlaryngectomy, speech failure, protective ptosis, entropion sphincter Odii dysfunction, pseudoachalasia, nonachalsia, oesophageal motor disorders, vaginismus, postoperative immobilisation tremor, bladder dysfunction, detrusor sphincter dyssynergia ,bladder sphincter spasm, hemifacial spasm, reinnervation dyskinesias, cosmetic use craw's feet, frowning facial asymmetries, mentalis dimples, stiff person syndrome, tetanus prostate hyperplasia, adipositas, treatment infantile cerebral palsy strabismus, mixed paralytic concomitant, after retinal detachment surgery, after cataract surgery, in aphakia myositic strabismus, myopathic strabismus, dissociated vertical deviation, as an adjunct to strabismus surgery, esotropia, exotropia, achalasia, anal fissures, exocrine gland hyperactivity, Frey syndrome, Crocodile Tears syndrome, hyperhidrosis, axillar palmar plantar rhinorrhea, relative hypersalivation in stroke, in Parkinsosn's, in amyotrophic lateral sclerosis spastic conditions, in encephalitis and myelitis autoimmune processes, multiple sclerosis, transverse myelitis, Devic syndrome, viral infections, bacterial infections, parasitic infections, fungal infections, in hereditary spastic paraparesis postapoplectic syndrome hemispheric infarction, brainstem infarction, myelon infarction, in central nervous system trauma, hemispheric lesions, brainstem lesions, myelon lesion, in central nervous system hemorrhage, intracerebral hemorrhage, subarachnoidal hemorrhage, subdural hemorrhage, intraspinal hemorrhage, in neoplasias, hemispheric tumors, brainstem tumors, myelon tumors. For details and symptoms see, e.g., Jost 2007, Drugs 67(5), 669 or Dressier 2000 in Botulinum Toxin Therapy, Thieme Verlag, Stuttgart, N.Y.

In another aspect of the invention, the composition is a cosmetic composition which can be formulated as described for a pharmaceutical composition above. For a cosmetic composition, likewise, it is envisaged that the compound of the present invention is in an aspect used in substantially pure form. Cosmetic compositions are, in a further aspect, to be applied intramuscular. In an even further aspect of the invention, cosmetic compositions comprising the neurotoxin can be formulated as an anti-wrinkle solution.

All references cited in this specification are herewith incorporated by reference with respect to their entire disclosure content and the disclosure content specifically mentioned in this specification.

### FIGURES

FIG. 1 is a schematic drawing of two embodiments according to the present invention (A and B). (A) shows a neurotoxin polypeptide with light chain (1) and heavy chain (2) with a linker comprising an epitope tag (4) flanked by two protease recognition and cleavage sites (3). (B) shows a neurotoxin polypeptide with light chain (1) and heavy chain (2) with a linker comprising an epitope tag (4) within a coiled coil structure (5) flanked by two protease recognition and cleavage sites (3).

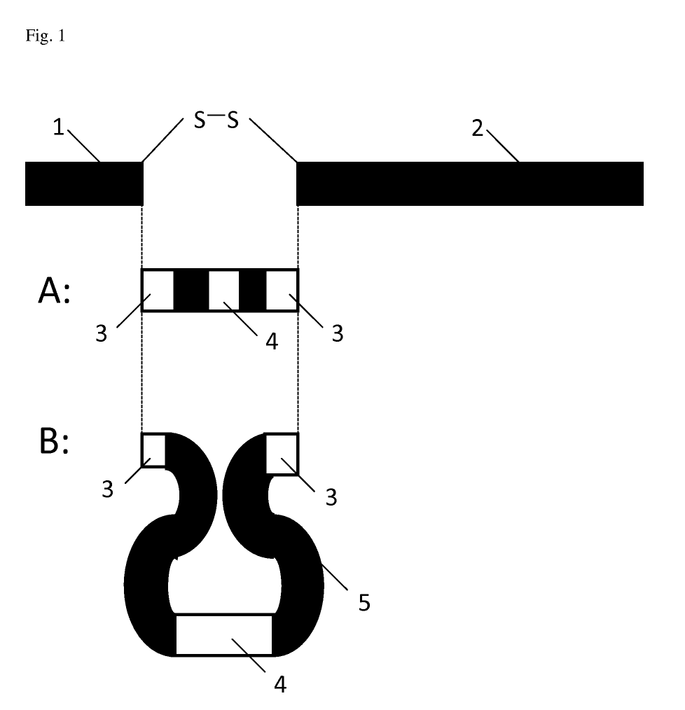

1-21. (canceled)

22. A polynucleotide encoding a neurotoxin polypeptide comprising a light chain, a linker and a heavy chain, wherein the linker is a modified linker comprising a heterologous amino acid sequence which confers at least one physicochemical property to the polypeptide which allows for separation of partially processed or unprocessed neurotoxin polypeptides from processed neurotoxin polypeptides, wherein the heterologous amino acid sequence is flanked N and C-terminally by a protease recognition and cleavage site.

23. The polynucleotide of claim 22, wherein the heterologous amino acid sequence binds with high affinity to a purification matrix.

24. The polynucleotide of claim 22, wherein the heterologous amino acid sequence comprises a purification tag.

25. The polynucleotide of claim 22, wherein the heterologous amino acid sequence increases the molecular weight of the neurotoxin polypeptide whereby the partially processed and/or unprocessed neurotoxin polypeptides can he physically separated from processed neurotoxin polypeptide.

26. The polynucleotide of claim 22, wherein the heterologous amino acid sequence comprises (i) at least one amino acid domain conferring high affinity binding to a purification matrix and (ii) at least one amino acid domain which brings cysteine residues forming an intramolecular disulphide bridge between light and heavy chain in close proximity and/or prevents the heterologous amino acid sequence from interfering with the formation of proper disulfide bonds between the light and heavy chain.

27. The polynucleotide of claim 26, wherein the at least one domain conferring high affinity binding to a purification matrix comprises a purification tag.

28. The polynucleotide of claim 26, wherein the at least one amino acid domain which brings cysteine residues forming an intramolecular disulphide bridge between light and heavy chain in close proximity forms a three dimensional structure which brings the cysteine residues forming the intramolecular disulphide bridge between light and heavy chain in physical proximity.

29. The polynucleotide of claim 28, wherein the three dimensional structure is formed by the C- and N-terminal amino acids of the heterologous amino acid sequence and is in the form of an antiparallel coiled coil or an antiparallel beta-sheet structure.

30. The polynucleotide of claim 26, wherein the heterologous amino acid sequence further comprises at least one detectable marker amino acid sequence.

31. The polynucleotide of claim 30, wherein the detectable marker amino acid sequence is selected from the group consisting of an amino acid sequence of a fluorescent protein, an amino acid sequence of an enzyme capable of generating a detectable signal, and an amino acid sequence of a detectable tag.

32. The polynucleotide of claim 24, wherein the purification tag is selected from the group consisting of a His-tag, a Myc-tag, a FLAG-tag, a strep-tag, a MBP-tag, a NusA tag, a GST-tag, streptavidin and avidin.

33. The polynucleotide of claim 22, wherein the protease recognition and cleavage site is selected from the group consisting of a neurotoxin light chain protease recognition and cleavage site from SNAP 25, a recognition and cleavage site from an E. coli protease, a Thrombin recognition and cleavage site, a Factor X recognition and cleavage site, a Trypsin recognition and cleavage site, an Enterokinase recognition and cleavage site, a TEV protease recognition and cleavage site, an HRV 3c recognition and cleavage site, and a PreScission Protease recognition and cleavage site.

34. The polynucleotide of claim 22, wherein the light and heavy chain of the neurotoxin polypeptide are the light and heavy chain of a neurotoxin selected from the group consisting of BoNT/A, BoNT/B, BoNT/C1, BoNT/D, BoNT/E, BoNT/F, BoNT/G and TeNT.

35. A vector comprising the polynucleotide of claim 22.

36. A host cell comprising the polynucleotide of claim 22.

37. The host cell of claim 36, wherein the host cell is a bacterial cell.

38. A host cell comprising the vector of claim 35.

39. The host cell of claim 38, wherein the host cell is a bacterial cell.

40. A polypeptide encoded by the polynucleotide of claim 22.

41. A method for the manufacture of a processed neurotoxin polypeptide comprising the steps of:
a) contacting an unprocessed neurotoxin polypeptide of claim 40 with a purification matrix under conditions and for a time sufficient for allowing binding of the the unprocessed neurotoxin polypeptide to the purification matrix;
b) contacting the unprocessed neurotoxin polypeptide bound to the purification matrix with a protease which is capable of cleaving the neurotoxin polypeptide at the protease recognition and cleavage sites resulting in processed and partially processed and/or unprocessed neurotoxin polypeptides; and
c) removing the released, processed neurotoxin polypeptide from the purification matrix.

42. A method for the manufacture of a processed neurotoxin polypeptide comprising the steps of:
a) contacting a neurotoxin polypeptide of claim 40 with a protease which is capable of cleaving the neurotoxin polypeptide at the protease recognition and cleavage sites;
b) contacting the processed, unprocessed and/or partially processed neurotoxin polypeptide obtained in step a) with a purification matrix under conditions and for a time sufficient for allowing binding of the the unprocessed and/or partially processed neurotoxin polypeptide to the purification matrix; and
c) removing the released, processed neurotoxin polypeptide from the purification matrix.

43. The method of claim 42, wherein step a) is carried out in a host cell or a lysate thereof' expressing the neurotoxin polynucleotide and a protease which is capable of cleaving the neurotoxin polypeptide at the protease recognition and cleavage sites.

44. A method for the manufacture of processed neurotoxin polypeptide comprising the steps of:
a) contacting a neurotoxin polypeptide of claim 40 with a protease which is capable of cleaving the neurotoxin polypeptide at the protease recognition and cleavage sites;
b) subjecting the processed, unprocessed and/or partially processed neurotoxin polypeptide obtained in step a) to physical conditions for a time sufficient for allowing physical separation of partially processed or unprocessed neurotoxin polypeptides from processed neurotoxin polypeptides; and
c) removing processed neurotoxin polypeptide after the physical separation.
